(19) United States
(12) Patent Application Publication
(10) Pub. No.: US 2012/0022374 A1
(43) Pub. Date: Jan. 26, 2012

(54) PULSE WAVE GENERATOR

(76) Inventor: Gosbert Weth, Bad Kissingen (DE)

(21) Appl. No.: 13/124,629

(22) PCT Filed: Oct. 18, 2008
(86) PCT No.: PCT/DE08/01707

§ 371 Date: Oct. 11, 2011

(51) Int. Cl. A61H 1/00; A61B 8/00

(52) U.S. Cl. 600/439; 601/15

(57) ABSTRACT

A pulse wave generator includes a housing having a pressure field generator from which individually controllable pressure pulses can be emitted, a pressure pulse focusing unit by which the pressure pulses can be focused at the focusing point in, or on, a body of a living being, a pressure coupler, from which the pressure pulses can be transmitted to the body, a controller for the intensity and point in time of each pressure pulse, at least one further field generator being integrated by which the individually controllable field pulses can be generated in the focusing point and the intensity and time of each pulse can be controlled.

The invention relates to a pulse wave generator, comprising a housing having a pressure field generator from which individually controllable pressure pulses can be emitted, a pressure pulse focusing unit by means of which the pressure pulses can be focused at a focusing point in or on a body of the living organism, a pressure pulse coupler, from which the pressure pulses can be transmitted to the body, and a controller for the intensity and point in time of each pressure pulse, at least one further field generator being integrated, by means of which the individually controllable field pulses can be generated in the focusing point and the intensity and point in time of each pulse can be controlled.

In the prior art, the introduction of pulses into the body of living organisms is widely known as a therapeutic measure. An example is so-called Atlas therapy, which goes back to the French doctor Arien (see “manuelle Medizin”, Volume 27, Issue 4, 1980 Edition, page 62, Springerverlag). In Atlas therapy, pains in the spinal column are treated by means of manual pulses, in particular light taps with the finger on the transverse processes of the first cervical vertebra. Moreover, tensions in the spinal column can be reduced or even eliminated. Even the first symptoms of multiple sclerosis can be improved, so that, e.g. an acute remission of paralysis is possible.

A considerable disadvantage is that the measurement of the pulses requires great experience and routine by the doctor performing it.

Another, known example for the successful effect of shock-like pulses is lithotripter, which serves for disintegrating kidney stones and gall stones. It consists of a pressure-field generator, which emits the individual pressure pulses, which behave like sound waves and are therefore focused in an acoustic lens on a focusing point in the body of the patient to be treated. A pressure pulse coupler, also known as an acoustic coupler, transmits the pressure pulses from the lithotripter to the patient's body. This coupler usually consists of a liquid-filled, permanently flexible bag, which is open at one side to the acoustic lens—called the pressure pulse focusing unit here—and is fixed at the outer edge thereof.

The pressure pulse focusing unit and the pressure pulse coupler ensure that most of the mechanical energy is focused at the focusing point. The lithotripter is oriented with the aid of an ultrasound diagnosis unit or an X-ray unit such that the focusing point is in a gall stone or kidney stone. If the orientation of the device is fixed, the intensity and frequency of the pressure pulse waves is slowly increased and the kidney stone or gall stone is successively divided into ever smaller fragments until, in an ideal case, it is pulverized. On the screen of the diagnosis unit, it can be followed how the initially solid block is gradually broken up into a diffuse cloud.

Although the lithotripter can gradually smash stones, it cannot convey them away. This final activity is performed by the body itself by conveying away the pulverized fragments together with the urea in “the normal way”.

The lithotripter is therefore a very comprehensible example of how shock waves that act on a particular, limited region of the human body, and there encounter a stationary, unwelcome configuration, which is therefore assessed as pathological, bring the stationary arrangement in this area into disarray, for example by the disintegration of stones, and only thereby permit access by the body's own systems, so that the original order can then be restored.

The lithotripter is a telling example for the generation of a mechanical field by a pressure-field generator.

However, in medical prior art, there are also devices known which, as therapy, apply a shock-like electrical field to the point of the complaints. An example of this is the defibrillator. It consists of a capacitor, which is suddenly discharged, so that, via two electrodes on the human chest, a very high electrical potential—up to 750 volt—is applied to the body and therefore a current flows via the body, whose strength depends on the respective body resistance, which on average is usually 50 ohm, so that a current up to 15 ampere is established. Because the capacitor only stores a limited amount of energy of typically 150 joule, the voltage and current intensity fall off very quickly, so that no permanent damage to other body regions and body parts, which are not actually affected, and to the heart itself is sustained.

The defibrillator is used principally for “ventricular fibrillation”, the cause of about 85% of all sudden cardiac deaths, in which via an electrically circulating excitation in the myocardial cells, the cells of the heart, the regular beat thereof is blocked. The electrical pulse wave of the defibrillator depolarizes a large number of cells simultaneously, so that it can no longer be excited for a relatively long time. This “refractory time” usually takes about 250 milliseconds. Then the stopped heart can be caused to beat again with its normal frequency by a heart-lung massage.

The common feature of both processes is that the shock-like pulse wave of a highly undesirable and steady state is at least at first “only” moderately changed. Even this new state is not yet that actually desired, but it has the very decisive advantage that now the body's own repair and rescue mechanisms can again act on the affected area and permit the return to a desirable normal state. In a highly reduced description, the pulse waves can also be classified as “helping the body to help itself”.

The advantages of this principle include the fact that only relatively short and inexpensive preparations are necessary for the patient and the costs of the equipment, if multiply used, are very low for an individual application case. The most important advantage is that the intervention does not cause side effects or collateral damage.

For example, the removal of kidney stones or gall stones can also be performed surgically. However, the wounding of the patient and weeks-long healing process contrast very sharply with the time for kidney stone disintegration. The cost differences for treatment are also correspondingly dramatic.

Likewise, for intervention in the heart, surgical intervention is not only known, but also conventional, not however for ventricular fibrillation, since the patient would no longer be alive even if the operation were initiated as soon as possible.

The advantage of the treatments of inner organs and other spatially limited body regions struck by a disorder, which are described here as examples, is thus that the treatment is only unfolds the actually intended effect through adjacent organs and body structures.

A considerable disadvantage is that the equipment can only be configured and used for a very specific disease or a very specific problem case. For example, EP 0 301 360, Frank Grasser, describes a lithotripter that is only optimized for gall stones. It is not even mentioned as applicable to kidney stones, let alone for other undesirable accumulations or concentrations in the body.

The U.S. Pat. No. 5,110,832, Ravi Xavier, describes an epidural catheter that is pushed between two vertebrae of the spinal column into the peridural space, that is to say very close to the spinal cord. In addition to an anaesthetic, electrical pulse with a voltage of up to 10 volt and a frequency of up to 120 hertz are emitted by means of externally attached electrodes in the region of the spinal cord. These pulsed electrical fields attenuate the patient's pain sensations significantly, so that the required dose of painkillers can be significantly reduced. The anaesthetic effect extends for longer operations and interventions, such as caesarean sections. This example shows a further application possibility for pulse waves of an electrical field.

A further known example of pulse waves of a mechanical force field is disclosed in U.S. Pat. No. 3,396,721, Mencacci. Here a piston is moved linearly through an electromagnet with a frequency of 5 to 15 times per minute. These mechanical beats are applies externally to the spinal column in the region of the lumbar vertebrae and ensure an activation of peristalsis, by initiating addition movement pulses to the nerve system of the gut muscles.

A known example of the application of pulse waves of a mechanical field is disclosed in U.S. Pat. No. 3,861,383, Kovach, with a skin treatment and skin massage unit, which discharges liquid from a nozzle onto the skin pulsating with a frequency of about 30 to 55 hertz, the range of action being limited by a cylinder, which is placed on the skin. Via the excitation of the blood flow, the ageing process of the skin is slowed and the formation of wrinkles is delayed. A disadvantage of the appliance is the large amount of discharged water, which must be intercepted or removed. It therefore requires the application of relatively long preparations and follow up work. Another restriction is that the appliance is only suitable for the skin, that is to say for the surface of the body. Other applications are excluded.

Another known example of a pulse wave generator, which uses a mechanical field, is disclosed in U.S. Pat. No. 4,265,228, Paul M. Zoll. It describes pistol-shaped housing, in the barrel of which a mass of an electromagnet is accelerated until it emerges from the barrel. The opening of the barrel is placed on the patient's chest and then the mass is accelerated with a preselected frequency and a preselected intensity until it impinges on the chest and is then brought back to the starting position by a spring.

The advantage of this device is that the frequency and impact intensity are given with considerably higher frequency, in comparison to a manual pressure massage, so that the pulses are sufficient for the desirable influencing of the heart beat but on the other hand are not so great that, for example, ribs could be broken. It is advantageous that, in its functional principle, the appliance is similar to a known nail gun, and can therefore be produced at comparatively very low costs, and is therefore readily available.

As a side effect, with relatively long use, a certain skin reddening is observed but regresses relatively rapidly. The crucial advantage, however, is that except for the application as heart pacemaker, no other applications are demonstrated.

The aforementioned known examples in the prior art show that sudden-acting pulses of a mechanical or electrical field have an indirect healing effect in a particular limited region of the body of a living organism if the nature of their field—mechanical or electrical and the intensity and duration and point in time of their respective pulse are tailored very accurately to the disorder to be treated.

Furthermore, the application of pulses of a magnetic field medical technology are known in in the prior art. U.S. Pat. No. 1,962,565, Lakhovsky, describes a coil, which is directed at or placed on the body, and is supplied with current pulses by a cyclic electrical energy source and a capacitor.

U.S. Pat. No. 5,556,418, Pappas, also describes an electrically energized coil on the patient's body. The current pulses for supplying this coil are actuated with even steeper flanks, so that even higher magnetic field intensities arise in the patient's body. For this arrangement, too, it is demonstrated that pulse waves have a healing effect by the change of field intensity if they are adapted to the defect to be treated within a narrow range in each case.

EP 0 683 657 of the inventor explains a source of “pulse-like waves”, which are restricted to the treatment of pain states or of the vegetative nervous system. The generation of a pressure pulse and the pain-alleviating or pain-soothing effects that can be achieved thereby are described in detail. As an alternative to the pressure pulse, an electrical pulse or an electromagnetic wave for pain therapy is administered. A clinical effect is thereby demonstrated.

The considerable restriction of this patent is that the appliance is only suitable for the treatment of pains and pain states, and therein generally only combats the symptoms of a diseases, but not its cause, apart from the relatively few cases, for example, of a phantom pain.

German Offenlegungsschrift DE 10 2006 024 701 discloses a combination of a mechanical and an electrical pulse source, both of which act on the heart. In particular, the reanimation of a heart that is no longer beating is described in detail. Apart from the heart, the possibility of acting on other organs is mentioned very generally.

A considerable disadvantage is that, apart from the heart, no single other organ is mentioned concretely. There is therefore a lack of any references to in which other organs which effect could be achieved.

Likewise there is a lack of any reference to the dimensioning and limitation of the pulse sources. It is only recommended “to reduce the radiated mechanical and electrical energy to a necessary minimum,” to largely exclude any possible side effects. It remains up to the user to find out how high the voltage and the current should be, and how often and how long a current should flow.

Appliances for treating a patient either by mechanical or electrical or magnetic pulses are thus known in the prior art. Also known is an appliance that combines mechanical and electrical pulses.

Against this background, the invention has the set itself the object of developing an appliance for treating a limited body region with a combination of pulsed waves, which can be used at as many organs as possible and at as many other points of the body as possible, and can be adjusted to as many different diseases as possible, in particular also cancer and as many types of pain and painful location, without affecting or even damaging other regions.

As a solution, the invention presents the fact that, as field generator for generating a magnetic field, a coil is used, which by discharging a capacitor charged up to 50 kV via a plasma chamber or via a spark gap, is energized by a very high current pulse of up to 3 kA, as a result of which it emits an electromagnetic pulse, whose energy content is briefly very high, and which, by induction, briefly raises the effective transmembrane potential of a tumour cell or another strongly irritated cells to a normal value.

It is thus a very essential gist of the invention that, for acting on that region of the body to be treated, a mechanical field and a magnetic field are chosen, and both fields act in a pulsed manner on the affected area. A further very important feature is that both types of field are oriented to a common focusing point, that is to say do not have any action at all in unaffected areas.

That also includes the fact that, in the case of zones lying deeper in the body, the distance from the outer surface of the body to the focusing point can be overcome by the acting fields also without side effects. The two acting fields, namely a mechanical and a magnetic, are thus not only additively superimposed but are both oriented to a very specific focusing point. As a result, its energy is concentrated in this point, which increases the healing effect, and remains away from other regions, which reduces any effect on other regions to the extent that no permanent effect remains.

A pulse wave generator according to the invention thus basically consists of two field generators, which in each case generate a field pulse, which acts on the focusing point and the intensity and point in time thereof can be controlled.

The first of the two field generators is always a pressure field generator whose pressure pulses are focused by a pressure pulse focuser at the focusing point or on the body of the living organism. To transmit from the pulse wave generator to the body, a pressure pulse coupler is used, and to control the intensity and the point in time of pulses of the two field generators, a controller is used.

A pulse wave generator according to the invention has, in addition to the above-described pressure field generator, a further field generator, which is designed as a magnetic field generator. It is a coil, which, as a result of the discharge of a capacitor charged at a very high voltage via a plasma chamber or spark gap, is energized by a very high current pulse. In practice, voltages up to 50 kV are present on the capacitor. Current pulses of up to 3 kA are generated in the coil.

This results in an electromagnetic pulse whose energy content is briefly very high, so that it can overcome corresponding barriers in the body. A very interesting example of this is the so-called transmembrane potential of a cell. In healthy cells it is about 80 to 100 mV, in cells highly irritated, for example by chronic stress, it is about 60 to 80 mV, but in the case of tumour cells it is only 15 to 20 mV.

The high value of the transmembrane potential of a healthy cell, at the extremely low dimensions of the cell of the order of several angstroms corresponds to a potential gradient of about 1,000 kV per meter. This high number explains the insensitivity of the healthy human cell to the very high electrical potentials that act on them.

It is assumed that the above-mentioned electromagnetic alternating field, by induction, raises the effective transmembrane potential of the tumour cell briefly to a normal value, and thereby allows access by normal repair mechanisms of the body.

This capability distinguishes the appliance according to the invention and therapies performed thereby from the most other disclosed devices and treatments of carcinomas, which only influence the interior of the cell and its DNA. A pulse wave generator according to the invention, on the other hand, also influences the cell membrane and the cell matrix that surrounds it.

Tumour cells are surrounded by a protective sheath of a hyaluronan fibrin layer as part of their cell matrix, which appears to ward off, for example, macrophages from attacking the tumour cell by feigning a correct physical healing process.

The third and further field generators can generate either a magnetic or an electrical or an electromagnetic field.

These three possible embodiments should not be confused with the fact that each current-carrying conductor builds up a magnetic field around itself, and that in principle both are active in generating the field within the appliance. It is rather decisive which type of field acts outwardly from the pulsed wave generator and into the body of the patient to be treated.

Here, it is to be distinguished that either only a magnetic field or only an electrical or an electromagnetic field is effective. A purely magnetic field occurs, for example, between the two poles of a horseshoe magnet. It is therefore thoroughly conceivable that the two legs of a U-shaped sheet-metal pack of an electromagnet are arranged in the direct vicinity of the focusing point F. Another known embodiment is a ring magnet, which is built up around the body of the patient.

An electrical field is generated, for example, by two electrodes at a distance from one another, the focusing point F being arranged approximately in the centre. An electrical voltage can then be applied in each case to these two electrodes.

An electromagnetic field occurs when high-frequency alternating current flows through a coil, or if a current pulse with very steep flanks generates high-frequency components. As a result, an electromagnetic wave forms, which propagates through space independently of conductors.

In the simplest case, both field generators emit a pulse, the pressure field generator a pressure pulse and, for example, an electromagnetic field generator an electromagnetic pulse. Very good treatment successes were achieved for the wide variety of applications if a pressure pulse of up to 100 milliseconds duration was combined with an electromagnetic pulse of at most the same length.

A pressure pulse that acts on the ambient air is received as sound. An interesting embodiment of the pressure field generator is thus a sound source. A focusing unit of a sound wave is thus designated a mechanical lens or acoustic lens. The pressure pulses audible as sound waves are transmitted to the patient's body by means of an acoustic coupler or coupling bag—a flexible pouch filled with liquid. If an electromagnetic field is effective in the focusing point at the same time, tumours, thrombi and many other illnesses can be successfully treated thereby, which can be demonstrated by numerous treatment successes.

It is always necessary to adapt the location of the focusing point, the intensity and the point in time of each pulse to the body regions to be treated, and the disorder acting there, within very narrow limits. With the pulse wave generator according to the invention, diseases and disorders can be successfully treated whose occurrence cannot be satisfactorily explained by the present state of knowledge, for example cancer.

Therefore—at present—it is not possible to give a sufficiently accurate explanation of the effect for every disease successfully treated with the pulse wave generator according to the invention. Rather, the user must find out in numerous trials how big the number of pulses, their intensity and their time intervals is for each treatment, and at which intervals and how often this treatment must be repeated. For the combination of a pressure field generator with an electromagnetic field generator, however, numerous impressive treatment results are available for various diseases and disorders, which are presented following the description of possible appliance variants.

It is namely the aim of this invention to create a universally applicable treatment appliance with pulse waves, which is applicable for multiple diseases and makes use of the advantages of a rapidly deployable, non-operative and side-effect-free treatment method.

It is the object of the user, from a precise knowledge of the proper functioning and malfunctioning of the affected body area or of the affected organ, to derive which field pulses are employed for treatment. In the absence of this knowledge, however, that could be comparatively rarely the case. Much more often, the respective treatment parameters are experimentally determined in that the user gropes towards an optimum effect with series of tests. This method is ethically responsible to the extent that other, conventional methods known in the prior art are fully open to the patient, and no side effects occur due to additional treatment with the pulse wave generator.

Since, with such procedures, interactions with possible medication and other forms of treatment are conceivable, exclusive treatment with the pulse wave generator is of course to be preferred, since only then is it entirely clear that treatment success is to be attributed exclusively to the use of the pulse wave generator.

Since it is part of the object of the invention to provide the person to be treated with the largest possible number of treatment parameters, in an alternative embodiment of a pulse wave generator according to the invention, for each field the amplitude and/or the frequency can be chosen increasing and/or decreasing and/or oscillating beyond the connection time.

Another very useful aid for a pulse wave generator according to the invention is an ultrasound diagnosis unit, the sound head of which is arranged in the longitudinal axis of the pressure field generator, specifically such that the sound head projects through the pressure pulse focusing unit and through the pressure pulse coupler somewhat beyond the outer surface of the pressure pulse couple and in this way can be pushed somewhat into the patient's body. In addition, the sound head is can be pivoted about its longitudinal axis.

It is decisively important that the measurement plane of the sound head passes through the focusing point. Then, during the application of the pulse wave generator according to the invention, the treating doctor can see on the screen whether a disorder, such as a tumour cell in the breast, is actually registered by the pulse wave generator.

A pressure field generator according to the invention can be designed in various models. Advantageous is, for example, a permanently elastic membrane, which is clamped at a plurality of points around its circumference and, in its centre or close thereto, can be deflected by means of a mechanical pulse, which is approximately perpendicular to the plane of the membrane and is oriented to the focusing point. Such membranes are known, inter alia, from loudspeakers or horns on motor vehicles. In the aforementioned application cases, an electromagnet serves for moving the membrane.

For a pulse wave generator according to the invention, however, a piezocrystal, a pretensioned spring, a pneumatic or hydraulic pressure accumulator with a cylinder, the ignition of an explosive charge or the pivoting of a cam can alternatively be used as drive. Alternatively, the pressure field generator can also be a mass, which is movable in the direction of the focusing point and is deflected in this direction by means of a mechanical pulse.

Concrete treatment successes were obtained when the kinetic energy given off by the movable mass is at least 5 millijoules and a velocity of at least 3 meters per second is achieved. Then a mass of less than 0.1 gram can be sufficient.

In the simplest case, the movable mass is a ball, which is fired like a bullet. The energy consumed for firing from a pressure field generator is focused entirely on the ball, the ball, in this embodiment, is thus also its own focusing unit. When the ball meets the body, it rebounds if the elasticity of its surface is too low in ratio to its mass. But if the ball is very soft, e.g. of foam, the energy reflected on impact with the body is temporarily stored in the body itself by compression of the ball, and then also transferred to the body. In this manner the ball partly takes over the functionality of the pressure pulse coupler.

If a movable ball meets the body of a patient, the pressure pulse triggered thereby propagates in the body also in a spherical manner. The consequence is that a movable mass is particularly suitable as pressure field generator when the desired focusing point lies very close to the surface of the patient.

A certain focusing effect can be achieved by the fact than a hollow or depression is disposed on that side of the movable mass that faces the focusing point. In this case, it must be ensured that this side also always encounters the body. A suitable means for this is a piston on the end face of which the hollow or depression is formed. The mechanical pulses triggered from the edges of the hollow compress in the central axis of the hollow, by which means focusing is achieved. The result of this is that an advantageous embodiment for a movable mass is a ring that encounters the body at one flat side, that is to say which must be linearly guided.

An advantage of the ring is that it does not compress ambient air in its centre on impact. In order to compensate for this disadvantage in the case of a piston with a hollow on its face end, an air channel can be provided from the lowest point of the hollow to the outside of the piston. On impact of the piston and on compression of the skin, compressed air leaves the interior space of the hollow through this channel.

For a mechanically movable piston, too, it is an advantageous coupling of the pressure pulse when its end face consists of a permanently flexible bag that is filled with a transmission liquid. On impact, the bag conforms to the surface of the boy and the liquid contained therein transmits the pressure pulse to the body with only very low losses.

An important characteristic of the pulse wave generator according to the invention is the pressure pulse focusing. For this purpose, an acoustic lens is used, which consists of a hard and vibration-resistant material in the form of a disc, whose edge is clamped in the housing such that it is oriented transversely to the direction of the pressure pulse. The disc has on at least one side a concave depression, which is rotationally symmetrical to the direction of the pressure pulse.

Such a shape is also known from the field of optical lenses. The effect of an acoustic lens is similar but not light but sound is focused.

In the same way that an optical lens depends on the fact that the incident light ray can penetrate into it without reflection, in an acoustic lens, the sound must also be able to enter and exit as unhindered as possible, for which purpose a transmission liquid has full-area contact with the acoustic lens on both sides. Via the transmission liquid, the acoustic lens has contact with the pressure pulse coupler on one side and with the pressure field generator on the second side. Due to the shape of its surface, it focuses the incident pressure pulses in the focusing point. The cross-section of an acoustic lens and its functional principle are similar to an optical lens.

As pressure field generator, a movable mass has already been mentioned. Another embodiment is a flat membrane. This membrane can also be deflected by a mechanical pulse as described above. A very interesting embodiment for moving the membrane is an electrical direct drive, with which a particularly good and sensitive control of the pressure pulse is possible.

To this end the membrane must contain an electrically conductive material, such as copper or aluminium. At the side facing the body, it is adjacent to the transmission liquid in the housing. At the other side, it lies on an insulating film, which in turn lies on a spiral-wound flat coil, which is retained by a coil support. Via a high-voltage pulse generator it can be energized with high-voltage pulses. It thereby generates an electromagnetic field, which in turn generates in the conductive material of the membrane an eddy current, as a result of which a further electromagnetic field is produced, which is opposite in direction to the original field, as a result of which the membrane is repelled by the flat coil.

Since the field acts in a planar manner on the membrane, a very high sound pressure can thereby be briefly attained. In practice, with pressure field generators, successful treatments were achieved, which generated pressures of at least 0.6 to 6 dyn per square meter. To this end, a high-voltage pulse is necessary, the current intensity of which is greater than 1 kA, and the voltage of which is greater than 1 kV.

As a further additional influencing possibility, a pulse wave generator according to the invention can be equipped with an apparatus for infusion of liquid active substances. By this means it is possible to combine the effects of pulse waves of two different types of field with a chemical effect.

A pulse wave generator according to the invention can be set up for treating a very wide range of complaints or pain sensations in or on the body of living organisms, which are localized in a limited space.

This includes tumours in the breast, in the pancreas, in the brain, in the prostate or in another organ or region, or Creutzfeld-Jacob diseases or apoplexy or heart rhythm disturbances or heart attacks or interstitium or embolism or thrombosis or wound and bone healing disturbances or cerebral circulatory problems or cerebral metabolic disturbances such as Alzheimer's disease or prostate hypertrophies or hypertrophies and metaplasia of other organs or inflammations of organs or body structures or chronic inflammations such as multiple sclerosis in healing or macula degeneration or digestion problems or wrinkling of the skin or endocrine diseases of the kidney, liver or diabetes.

As an example, a successful tumour treatment of a patient is given, whose bladder, due to a 15 cm large tumour, was completely filled with tumour tissue, so that is could no longer take up urine. The tumour had already grown into the wall of the bladder. For pain therapy, the patient had to be administered with a morphine dose of 400 mg per day.

The patient was subjected to a series of 3,000 pulses from a pulse wave generator according to the invention with a pressure field generator and an electromagnetic field generator several times per week over a period of 2 months.

After this treatment, it could be shown by nuclear spin tomography that the tumour had completely regressed and the bladder wall had been reduced to its original wall thickness. The administration of morphine could be completely dispensed with. In nuclear spin tomography, as well as in the function, it could be demonstrated that the bladder was again filled with its normal urea volume. No side effects of the treatment could be recorded.

That is in distinct contradiction to the treatment method of chemotherapy that was formerly conventional, which causes massive damage to other, unaffected, organs and body regions, and still only makes survival possible for a portion of the patients.

As effect mechanism of the treatment with a pulse wave generator according to the invention, biochemical studies could show that anaerobic metabolism of the tumour cells could be further returned to aerobic metabolism during the treatment, that is to say to the same behaviour as “normal” cells. It can be assumed that, due to these changes, the tumour cells were again susceptible to the body's own “repair processes.”

Another application example is 20 female patients with a mammary carcinoma. In the case of 18 patients, the nodes were already bigger than 3 cm.

After treatment with a pulse wave generator according to the invention over 2 months with at least 3 pulse series per week, with 3,000 pulses in each case, the nodes had completely regressed. Through post-controls with MRT, it was found that, for that patient, no pathological findings were detectable even in the following 5 years. During treatment, the pain symptoms were continually reduced and did not return after discontinuation of the treatment, either as pain on pressure or as continuous pain.

Another field of application of a pulse wave generator according to the invention is indirect therapy of an inner organ via the associated vegetative nervous system. The time intervals between two successive pulses and pulse series were one to ten minutes. Via the afferent dorsal roots of the spinal nerves of an organ, not only afferent vegetative fibres are guided from the respective associated organ regions, but also from skin segments (dermatome). Every disease therefore contains oversensitive, painful skin areas, also known as head's zones. With the aid of these zones, therefore, the crucial ganglion can be determined for the respective disease and impacted with the pulse-like wave. The celiac inferior, cervical inferior, cervical superior, sacral and pelvinum ganglia have been successfully treated.

Alternatively, a head's zone itself, as a neutrally sensitive area, can be subjected to a pulse wave according to the invention. The associated organ is therapeutically influenced in both ways.

As evidence of the effect, a change of the level of neurotransmitters and biogenic amines should be observed in the entire organism. See W. Kloepfer and Weth, G. et al. “Atlas Therapy and Neurotransmitters in Patients Affected by Multiple Sclerosis”, manuelle Medizin, Volume 27, edition 4, page 82, August 1989, Springer Verlag.

Another efficient application of a pulse generator according to the invention is the treatment of pain, in particular as a result of tumours, rheumatoid diseases and their therapy or tension or blood circulation problems, e.g. of the legs as a result of metabolic disorders or heart attack and stroke, or vasospastic diseases or internal diseases, such as Whipple's disease, angina pectoris, stomach pains or functional epigastric complaints or neurological and psychiatric diseases, such as neuralgia, multiple sclerosis or diseases of the neurovegetative syndrome.

A pulse wave generator according to the invention can, by the choice of a suitable focusing point and corresponding setting of the intensity and time of each field pulse, even be adjusted to the modulation of the metabolism of the cell, specifically to the mitochondrial, cellular or extracellular metabolism or the cell matrix.

In the application of the pulse generator according to the invention, it has proved particularly effective if each individual pulse has a duration of a maximum of 0.1 seconds. The best effect was obtained with a single pulse having a total duration of 70-100 milliseconds.

For many diseases, illnesses and pains, the following treatment plan proved most effective: Up to 10,000 individual pulses of the field generators in each case act at a distance of at most 0.5 seconds on that focusing point of the body that is to be treated. This pulse series was repeated at an interval of one day up to one week over a period of up to 3 months.

As a variant for some applications, each individual pulse was expanded to a pulse series of up to 100 milliseconds duration.

By means of a pulse wave generator, the matrix of a non-healthy cell can—as mentioned—be modulated such that its extracellular metabolism approaches that of a healthy cell, which is noticeable as improved blood circulation, which can be demonstrated through an increase of the dopamine content. Dopamine is one of the most important neurotransmitters, particularly in cerebral metabolism.

In the case of cells that have been damaged by a stroke, heart attack or tumour disease, it could be measured that the dopamine level before treatment with a pulse wave generator according to the invention was significantly lower than after the treatment.

Another indication of the positive effect of the pulse wave generator according to the invention is the dramatic reduction of the daily vomiting—as is typical of carcinoma patients who are subject to chemotherapy or radiotherapy—before and after pulse wave treatment.

Further details and features of the invention are explained below in greater detail with reference to an example. This is not intended to restrict the invention, but only to explain it.

In schematic view,

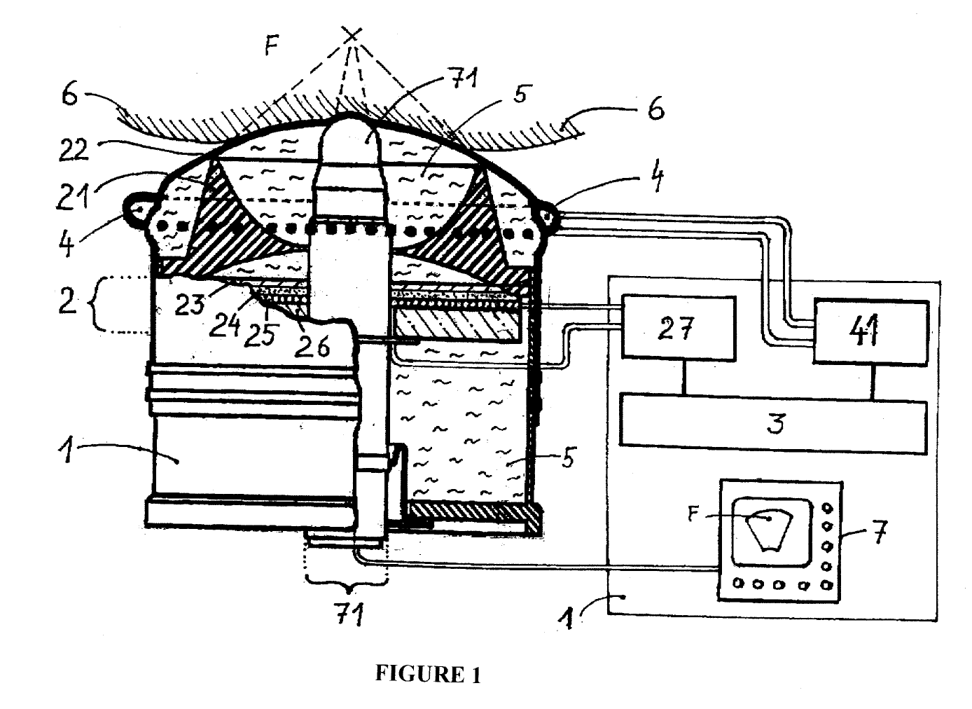

FIG. 1 shows a longitudinal section through a pulse wave generator according to the invention

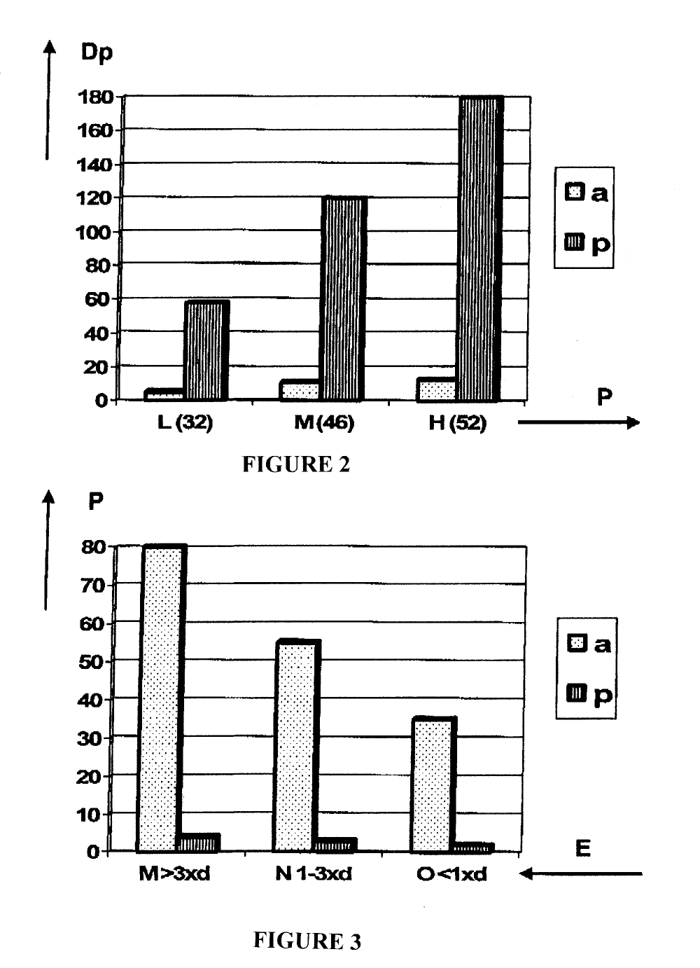

FIG. 2 Percentage increase of dopamine as a result of the pulse wave treatment of cells affected by strokes

FIG. 3 Reduction of daily vomiting of carcinoma patients as a result of the pulse wave treatment

FIG. 1 shows the housing 1 in two parts: In the right half, the housing 1 is illustrated only as a block circuit diagram with four electrical functional modules contained therein, at the left is shown a further part of the housing 1 as a realistic cutaway diagram, in which those modules are disposed that generate, focus and transmit the pressure field of the pressure field generator 2, and generate and radiate an electromagnetic field.

In the left, realistic half of FIG. 1, the approximately pot-shaped part of an—in this embodiment rotationally symmetrical—housing 1 is shown partly diagrammatically cutaway, so that the view of the pressure field generator 2 contained therein and the field generator 4 superimposed thereon, in this case for generating an electromagnetic field, is exposed. This portion of the housing 1 is filled with the transmission liquid 5, which transmit the pressure pulses from the pressure field generator 2 to the pressure pulse focusing unit 21—also known as an acoustic lens. Here they are focused on the focusing point F and transmitted to the body 6 by the pressure pulse coupler 22.

FIG. 1 shows clearly that the cross-section of the pressure pulse focusing unit is similar to the cross-section of a spectacle lens. This unit is therefore also known as an “acoustic lens”. The functional principle is also similar to that of an optical lens. Corresponding to the “refractive index” of the spectacle lens, which differs significantly from the surroundings,” the sound velocity within the solid material of the acoustic lens is significantly different from the velocity of sound in the transmission liquid 5—which surrounds the lens. As a result, the pressure pulses enter at a different angle to the surface than they exit from it.

The pressure pulses focused by the acoustic lens are transferred to the pressure pulse coupler 22, also known as the “acoustic coupler.” In FIG. 1, it can be readily seen that the pressure pulse coupler 22 consists of a—flexible—bag, which is filled with the transmission liquid 5. As a result the sound waves emitted by the pressure pulse focusing unit 21 are almost completely transmitted to the focusing point F in the body 6 of the patient. In FIG. 1 the pressure pulse coupler 22 can be seen in cross-section; its flexible outer layer is shown by a broad black line.

The progress of the pressure pulse waves to the focusing point F is shown with dotted lines. At this focusing point F, that region of the body affected by a disease or disorder must be adjusted. The edge zone of the body 6, shown partly hatched in FIG. 1, could thus represent a breast that is affected by a tumour, which grows at the position of the focusing point F.

In FIG. 1, as an exemplary embodiment of the pressure field generator 2, an electromagnetic pressure generator is shown. In the section, the membrane 23 can be seen, which contains electrically conductive ingredients. It is separated from the flat coil 25, which lies on the coil support 26, by the insulation film 24. The flat coil 25 in this case consists of an electrical conductor laid in a spiral on the coil support 26, which can be seen in cross-section as a series of circles. This coil is connected via two leads to the high-voltage pulse generator 27, which is only shown schematically.

On the realistically shown housing 1, the further field generator 4 can be seen. In this exemplary embodiment, as well as the pressure field generator 2, an electromagnetic field generator can be seen. It consists of an annular coil, which is mounted around the upper edge of the housing 1, which is also approximately circular here. At the left and right of the housing 1, the coil of the field generator 4 can be seen in section. The course of the coil beyond the housing 1 is shown with large dots and a broken line. This coil is connected via two electrical leads to the supply unit 41, which is only schematically shown in FIG. 1. This supply unit 41 supplies the coil with current pulses of very high current intensity and very high voltage. In FIG. 1, it can be readily seen that an electromagnetic field develops in the coil, which propagates towards the body 6 and penetrates the focusing point F. At the other side, it is reflected by the metallically conductive housing 1 and also guided into the body 6.

In FIG. 1, two field generators are thus drawn, in one case the mechanical pressure field generator 2 and in the other case the other field generator 4, shown here in the embodiment as an electromagnetic field generator. The supply units of both field generators and in one case the high-voltage pulse generator 27 and in the other case the current pulse generator for the coil, designated here the supply unit 41. Both are controlled by the controller 3.

It is not shown in FIG. 1 that the controller ensures that the pulses or pulse sequences have a length of at most 100 milliseconds and depending on the type of disorder or disease to be treated trigger the next pulse after typically 500 milliseconds pause and—depending on the disease or disorder—activate up to 10,000 pulses in sequence.

FIG. 1 shows, as further optional equipment of a pulse wave generator according to the invention, an ultrasound diagnosis unit 7 drawn in the schematically illustrated portion of the housing 1. The associated sound head 71 is arranged in the longitudinal axis of the housing 1, and is thereby also located in the longitudinal axis of the pressure field generator 2 and the further field generator 4.

The tip of the sound head 71 projects slightly beyond the flexible outer skin of the pressure pulse coupler 22. In addition, the sound head 71 is can still be slightly pivoted about its longitudinal axis with respect to the housing 1. By this means, on the monitor of the ultrasound diagnosis unit 7, the region is visible in which both field generators 2 and 4 are active, so that it can be checked whether the focusing point F actually meets the diseased area of the body, e.g. a tumour. To facilitate the work, the focusing point F is therefore marked on the screen of the ultrasound diagnosis unit 7.

In FIG. 2, the measured percentage increase of dopamine is shown by the cells damaged by pulse wave treatment in the case of stroke patients P as a diagram.

A set of 130 patients P were split into three groups dependent on their dopamine level Dp after pulse wave treatment, specifically into a group H, in which the dopamine level Dp is increased to about twice the normal value of 100% by the treatment. H stands for “high”. To this group, 52 patients P were assigned. The exact mean value of the dopamine level Dp of these 52 patients P in group H increased by the treatment to 180% of the normal value of 100%.

The dopamine level Dp of a second group L of patients P only had about half of the normal value of 100% even after the treatment. L here stands for “low”. To this group, 32 patients P were assigned. The exact mean value of the dopamine level Dp of these patients P increased to 58% of the normal value of 100% as a result of the treatment.

In the case of the 46 patients P, the dopamine level Dp after the treatment increased approximately to the normal value. This group is designated M for “medium”. The exact mean value of the dopamine level Dp of these patients P increased to 120% of the normal value of 100% as a result of the treatment.

In the first group L, the dopamine level of an average of 5.5% increased to 58% of the normal value, that is to say to 10.5 times the initial value. In the second group M, the dopamine level of an average of 11% increased to 120%, that is to say to 10.9 times the initial level. And in Group H, the relative increase from an average of 13% to 180% was highest at 13.8 times. In FIG. 2, on the horizontal axis, the respective number P of patients in the three groups L, M and H is shown. On the vertical axis, for each group, the average dopamine level Dp in the blood is drawn with respect to the normal value of the dopamine level Dp as a bar, specifically with “a” before and “p” after the treatment with a pulse wave generator according to the invention.

FIG. 2 shows that in the case of cancer treatment with a pulse wave generator according to the invention, the dopamine level Dp can be increased to at least 10 times the initial value, which indicates a dramatic step back to normal metabolism of the previously pathological cells.

FIG. 3 shows the reduction of daily vomiting E of carcinoma patients P as a result of the pulse wave treatment On the vertical axis, the daily frequency of vomiting E is plotted. Left the greatest frequency, right the lowest frequency. On the vertical axis, the number of patients P who suffer from such frequency of daily vomiting.

For the study, a set of 170 patients P is subdivided into three groups. To the group M, all 80 patients were assigned who had to vomit more than 3 times per day. The 55 patients of group N vomited 1 to 3 times per day and the 35 patients of the group did not vomit every day, that is to say less than once daily.

In FIG. 3, the number of patients before treatment with a pulse wave generator according to the invention is designated “a” and the number after treatment is designated “p”.

In the worst affected group M, the treatment reduces the number of patients P who still have to vomit so often from 80 to 4, that is to say by 95%. In the medium group M, the proportion of the patients who are still so badly affects is reduced from 55 to 3, that is to say by 94% and in the least affected group O from 35 to 2 patients P, that is to say also a reduction by 94%.

FIG. 3 thus shows impressively that treatment with a pulse wave generator according to the invention can also reduce the side effects of a carcinoma disease.

### LIST OF REFERENCE CHARACTERS

### Claims and FIG. 1

F Focusing point of the pressure field generator 2 in or on the body 6
1 Housing to accommodate the field generators 2 and 4
2 Pressure field generator in the housing 1
21 Pressure pulse focusing unit between the pressure field generator 2 and pressure pulse coupler 22
22 Pressure pulse coupler, between the pressure pulse focusing unit 21 and body 9
23 Membrane of the pressure field generator 2
24 Insulation film, between the membrane 23 and flat coil 25
25 Flat coil for generating the pressure pulse for repulsion of the membrane 23
26 Coil support, supports the flat coil 25
27 High-voltage pulse generator for activating the flat coil 25
28 Transmission liquid between the membrane 23 and pressure pulse focusing unit 21 and between pressure pulse focusing unit 21 and pressure pulse coupler 22
3 Controller of the field generators 2 and 4
4 Further field generator, generates a field in the focusing point F
41 Supply unit of the field generator 4
5 Transmission liquid between the membrane 23 and pressure pulse focusing unit 21 and between pressure pulse focusing unit 21 and pressure pulse coupler 22
6 Body with focusing point F, where the fields of the field generators 2 and 4 act
7 Ultrasound diagnosis unit
71 Sound head of the ultrasound diagnosis unit 7, integrates in the pressure field generator 2

### LIST OF REFERENCE CHARACTERS CONTINUED

### FIGS. 2 and 3

a Before the pulse wave treatment
p After the pulse wave treatment

### FIG. 2

Dp Dopamine level in % of the normal value
P Number of patients
L Patient group with relatively low dopamine level
M Patient group with medium dopamine level
H Patient group with high dopamine level

### FIG. 3

P Number of patients
E Frequency of vomiting
M>3×d Vomiting more than three times daily
N 1-3×d Vomiting 1-3 times daily
O<1×d Vomiting less than 1 times daily

1-28. (canceled)

29. A pulse wave generator, comprising:
a pressure field generator from which individually controllable pressure pulses are able to be emitted;
an additional pressure field generator;
a pressure pulse focusing unit for focusing said pressure pulses at a focusing point in, or on, a body of a living organism;
a pressure pulse coupler from which the pressure pulses are able to be transmitted to the body of the living organism; and,
a controller for controlling intensity and point-in-time of each said pressure pulse, said additional pressure field generator being integrated, so that the individually controllable field pressure pulses are generated in the focusing point and the intensity and the point-in-time are controllable, said additional pressure field generator for generating a magnetic field being a coil, which by discharging a capacitor charged up to 50 kV, is energized, via a plasma chamber or a spark gap, by a current pulse of up to 3 kA, thereby producing an electromagnetic pulse having an energy content that is very high for a short duration of time and, by induction, raises an effective transmembrane potential of a tumor cell, or a highly irritated cell, to a normal value for a brief duration of time.

30. The pulse wave generator according to claim 29, wherein the trans-membrane potential of a tumor cell is raised for a short duration of time from 15 mV to 20 mV to a transmembrane potential for a healthy cell of approximately 80 mV to 100 mV.

31. The pulse wave generator according to claim 29, wherein said additional pressure field generator is capable of generating a magnetic field, an electrical field or an electromagnetic field.

32. The pulse wave generator according to claim 29, further comprising a sound head of an ultrasound diagnosis unit disposed in a longitudinal axis of said pressure field generator, said sound head projecting through said pressure pulse focusing unit and said pressure pulse coupler beyond an outer surface of said pressure pulse coupler with a measurement plane of said sound head running through the focusing point.

33. The pulse wave generator according to claim 29, wherein said pressure field generator is an elastic member clamped on its circumference and, in a center of said elastic member, is able to be deflected via a mechanical pulse oriented approximately perpendicularly to a surface of said elastic membrane and the focusing point.

34. The pulse wave generator according to claim 33, wherein said mechanical pulse is generated via an electromagnet, a piezocrystal, a pretensioned spring, a pneumatic or hydraulic pressure accumulator having a cylinder, an ignition of an explosive charge or a pivoting of a cam.

35. The pulse wave generator according to claim 29, wherein said pressure field generator is a mass movable toward the focusing point and is deflected by a mechanical pulse toward the focusing point.

36. The pulse wave generator according to claim 35, wherein said mass emits a kinetic energy of at least 5 millijoules.

37. The pulse wave generator according to claim 35, wherein said mass has a depression, or a hollow area, facing the focusing point serving as said pressure pulse focusing unit.

38. The pulse wave generator according to claim 29, wherein said pressure pulse coupler comprises a flexible bag tilled with a transmission liquid, said flexible bag being adjacent to the body of the living organism at a first side and, at a second side, being adjacent to said pressure pulse focusing unit.

39. The pulse wave generator according to claim 29, wherein said pressure pulse focusing unit is an acoustic lens comprising a hard and vibration-resistant material formed as a disk having an edge that is oriented transversely to a direction of the pressure pulse and having on at least one side of a concave depression shaped so that it is rotationally symmetrical to the direction of the pressure pulse and in contact with a transmission liquid, wherein, at a first side, fills said pressure pulse coupler and, at a second side, is adjacent to said pressure, said disk focusing a pressure pulse from said pressure field generator in the focusing point.

40. The pulse wave generator according to claim 29, wherein said pressure field generator comprises a flat membrane containing an electrically conductive material.

41. The pulse wave generator according to claim 29, wherein said pressure field generator generates a pressure of at least 0.6 dyn/mm³.

42. The pulse wave generator according to claim 29, wherein said pressure field generator generates a pressure of at least 6 dyn/mm³.
